(19) United States
(12) Patent Application Publication
(10) Pub. No.: US 2010/0063118 A1
(43) Pub. Date: Mar. 11, 2010

(54) Hydantoins Having RNase Modulatory Activity

(75) Inventors: Matthew W. Olson, Bardonia, NY (US); Martin Di Grandi, Harriman, NY (US); Amarnauth Prashad, New City, NY (US)

Correspondence Address: FITZPATRICK CELLA (WYETH LLC), 1290 Avenue of the Americas, NEW YORK, NY, 10104-3800, US

(73) Assignee: Wyeth, Madison, NJ (US)

(21) Appl. No.: 12/501,133

(22) Filed: Jul. 10, 2009

Related U.S. Application Data
(62) Division of application No. 11/076,995, filed on Mar. 11, 2005, now abandoned.
(60) Provisional application No. 60/552,530, filed on Mar. 12, 2004.

(51) Int. Cl. A61K 31/4164; C07D 233/72; C12N 9/99; A61P 31/12; A61P 31/18; A61P 35/00; C12Q 1/68; C07D 471/10; C07D 471/04

(52) U.S. Cl. 514/389; 548/317.1; 548/319.5; 435/184; 435/6; 546/20; 546/82

(57) ABSTRACT

The present invention relates to hydantoin derivatives having RNase H, polymerase and/or HIV reverse transcriptase modulatory, and particularly, inhibitory activity. Included in the invention are the hydantoin derivatives, compositions containing the derivatives, methods of synthesis of the derivatives, screening methods to identify the derivatives, and methods of treatment using the hydantoin derivatives, including the treatment of HIV, AIDS and retrovirus-associated cancer.

This application claims the benefit of U.S. Provisional Patent Application No. 60/552,530, filed Mar. 12, 2004.

### BACKGROUND OF THE INVENTION

1. Field of the Invention

The invention relates to the use of hydantoin derivatives in the manufacture of anti-HIV pharmaceuticals, retrovirus-associated cancer pharmaceuticals, reverse transcriptase modulators, polymerase modulators, RNase modulators, and to certain novel hydantoin compounds and to processes for the preparation of and compositions containing such novel compounds.

2. Related Background Art

The retrovirus designated human immunodeficiency virus (HIV) is the etiological agent of the complex disease that includes progressive destruction of the immune system and degeneration of the central and peripheral nervous system (acquired immune deficiency syndrome; AIDS). A common feature of retrovirus replication is reverse transcription of the RNA genome by a virally encoded reverse transcriptase. Reverse transcriptase is implicated in the infectious lifecycle of HIV, and compounds such as nucleoside and non-nucleoside reverse transcriptase inhibitors, which interfere with the function of this enzyme, have shown utility in the treatment of conditions including AIDS.

Presently, there are four categories of drugs used to treat HIV infection, which include nucleoside analogue reverse transcriptase inhibitors, non-nucleoside reverse transcriptase inhibitors and protease inhibitors. Reverse transcriptase inhibitors, including the nucleoside and non-nucleoside categories, interfere with HIV reverse transcriptase, which, as noted above, is required for viral replication. Protease inhibitors interfere with the enzyme protease, which plays a major role in viral infection. Forms of anti-HIV therapy include giving only one reverse transcriptase inhibitor at a time (monotherapy), a combination of two or more reverse transcriptase inhibitors (combination therapy), and a combination of reverse transcriptase inhibitors and protease inhibitors (combination therapy with protease inhibitors). Nucleoside analogues include AZT (zidovudine, Retrovir), ddI (didanosine, Videx), 3TC (lamivudine, Epivir), d4T (stavudine, Zerit), abacavir (Ziagen) and ddC (zalcitabine, Hivid). AZT and 3TC are also available in a single combined pill called Combivir and AZT, 3TC and abacavir are available in a single combined pill called Trizivir. Tenofovir (Viread), a nucleotide analogue, is the only nucleotide analogue currently available for prescription and is only licensed to give people on their second or later treatment combination, although it may be given to people in their first-line treatment. Nucleotide analogues are very similar to nucleoside analogues. The only difference is that nucleotide analogues, unlike nucleoside analogues, are chemically preactivated and thus require less processing in the body for them to become active. Non-nucleoside reverse transcriptase inhibitors include Sustiva, nevirapine (Viramune), and delavirdine (Rescriptor).

Many of the treatments which inhibit reverse transcriptase activity that are currently available, particularly the nucleoside analogues, are associated with serious side effects and require long term treatment to be effective. In addition, the virus is able to mutate in response to the drugs and becomes resistant to them. Therefore, there is a constant need to provide new and better treatments for HIV and AIDS and particularly new drugs that inhibit HIV reverse transcriptase.

Other treatments that are being developed which relate to inhibiting other pathways of the viral life cycle include, for example, U.S. Pat. No. 5,767,140, which discloses a series of 5,5-disubstituted hydantoin derivatives which can inhibit HIV-induced death and virus production in mammalian cells by targeting the viral-encoded glycoprotein, gp120 which has an affinity for the cellular CD-4 receptor of the host and which, like protease inhibitors, inhibits viral infection. Kim et al. describe hydantoin derivatives which are capable of inhibiting HIV infection based on their antagonistic activity of the human chemokine receptor CCR5, which is implicated in HIV entry into cells. See Kim et al., Biooroganic & Medicinal Chemistry Letters 11:3099-3102 (2001); and Kim et al., Bioorganic & Medicinal Chemistry Letters 11:3103-3106 (2001). Other hydantoin derivatives are generally described in U.S. Pat. Nos. 3,847,933; 2,600,835; 4,044,019; 5,338,859; 5,606,071; 5,859,190; 6,018,053; 6,344,564, 6,436,962; 6,320,055; 6,294,694; 5,608,076; 5,565,575; 5,516,908; 5,346,913; 5,326,878, 4,022,620; and U.S. Patent Publication No. 2003/0134856. There is, however, no disclosure or suggestion of using these hydantoins for the treatment of HIV.

Hydantoin derivatives have previously been shown to inhibit HIV replication. See U.S. Pat. No. 5,767,140; which discloses a series of 5,5-disubstituted hydantoin derivatives which can inhibit HIV-induced death and virus production in mammalian cells. However, the inhibition of HIV replication by the hydantoin derivatives described in U.S. Pat. No. 5,767,140 is not mediated by targeting HIV reverse transcriptase, but rather by targeting the viral-encoded glycoprotein, gp120 which has an affinity for the cellular CD-4 receptor of the host and which, like protease inhibitors, inhibits viral infection. The present invention for the first time describes hydantoin derivatives which inhibit HIV reverse transcriptase and are useful for treating and preventing HIV and AIDS.

### SUMMARY OF THE INVENTION

The present invention relates to the use of hydantoin derivatives which inhibit reverse transcriptase activity and more particularly, which inhibit the RNase H activity and polymerase activity, and its resistant varieties, and are modulators, especially inhibitors thereof, for the treatment and prevention of HIV and AIDS. In a preferred embodiment, the hydantoin derivatives inhibit the RNase H activity of HIV reverse transcriptase and the RNA dependent DNA polymerase activity of HIV reverse transcriptase. The hydantoin derivatives of the invention are also useful for treating retrovirus-associated cancer, such as adenocarcinoma of the breast. The invention includes hydantoin derivatives with RNase H and/or HIV reverse transcriptase modulatory, and particularly inhibitory, activity. Included in the invention are the hydantoin compounds, compositions containing the compounds, methods of synthesis of the compounds, and methods of treatment using the hydantoin compounds, including the treatment of HIV, AIDS and retrovirus-associated cancer. The compounds of this invention, as described herein, include prodrugs, pharmaceutically acceptable salts, and pharmaceutically active metabolites thereof.

An embodiment of the invention is a compound of formula I:

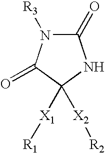

wherein R₁, R₂ and R₃ are independently selected from the group consisting of alkyl, alkenyl, alkynyl, aryl alkyl, alkylaryl, heterocycloalkyl, cycloalkyl, aryl, heteroaryl, any of which may be optionally substituted,

and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

In one preferred embodiment the compounds are of formula I where R₁ and R₂ are selected from the group consisting of phenyl, substituted phenyl, alkyl, alkylaryl, and arylalkyl. In another preferred embodiment the compounds are of formula I where R₃ is selected from the group consisting of phenyl and substituted phenyl.

In a further preferred embodiment, the hydantoin derivatives of the present invention are of formula I and are selected from the group consisting of the following compounds:
4-ethyl-4-(4-methylphenyl)-4H-imidazole-2,5-diol;
6-[{[{(2,5-dioso-4,4-diphenylimidazolidin-1-yl)acetyl]amino}(phenyl)acetyl]amino}-3,3-dimethyl-7-oxo-4-thia-1-azabicyclo[3.2.0]heptane-2-carboxylic acid;
5-methyl-5-phenyl-3-[(2E)-3-phenylprop-2-enoyl]imidazolidine-2,4-dione;
5,5-dimethyl-3-vinyl-imidazolidine-2,4-dione;
3-[2-(3-chloro-4-methylphenyl)-1-methyl-2-oxoethyl]-5-methyl-5-phenyl-2,4-imidazolidinedione;
3-[(2E)-3-(3,4-dichlorophenyl)prop-2-enoyl]-5-methyl-5-phenylimidazolidine-2,4-dione;
3-(3-bromopropyl)-5,5-diphenyl-2,4-imidazolidinedione;
(5R)-5-methyl-5-phenyl-3-{(2E)-3-[4(trifluoromethyl)phenyl]prop-2-enoyl}imidizolidine-2,4-dione;
di(tert-butyl) 8-benzyl-2,4-dioxo-1,3,8-triazaspiro[4.5]decane-1,3-dicarboxylate;
(5S)-5-methyl-5-phenyl-3-{(2E)-3-[4-(trifluoromethyl)phenyl]prop-2-enoyl}imidazolidine-2,4-dione;
3-{(2E)-3-[4-fluoro-3-(trifluoromethyl)phenyl]prop-2-enoyl}-5-methyl-5-phenylimidazolidine-2,4-dione; and
3-(4-chloro-3-methylbenzoyl)-5-methyl-5-phenylimidazolidine-2,4-dione.

In another preferred embodiment, the hydantoin derivatives of the present invention are selected from the group consisting of the following compounds:
(5S)-5-ethyl-5-(4-methylphenyl)imidazolidine-2,4-dione;
5-(3,5-dibromo-2-hydroxy-benzylidene)-imidazolidine-2,4-dione;
5-cyclopropyl-5-(3,4-dimethyl-phenyl)-2,4-imidazolidinedione;
5-(3,5-dichloro-2-hydroxy-benzylidene)-imidazolidine-2,4-dione;
2-benzoyl-10b-phenyl-6,10b-dihydro-5H-imidazo[5,1-a]isoquinoline-1,3-dione; and
5-(4-bromo-phenyl)-5-ethyl-imidazolidine-2,4-dione.

The compounds of the present invention can inhibit RNase and polymerase activity. In a preferred embodiment, the compounds inhibit HIV-reverse transcriptase and are useful in the treatment and prevention of HIV and AIDS. Accordingly, in one embodiment, the present invention provides a method for inhibiting RNase H nuclease activity comprising contacting RNase H with the hydantoin derivatives of the present invention in an amount sufficient to inhibit the RNase H nuclease activity. Inhibition of the RNase H nuclease activity by the compounds of the present invention is useful, inter alia, for the treatment and prevention of HIV, AIDS and retrovirus-associate cancer. In one embodiment, the RNase activity is the RNase activity of HIV reverse transcriptase. Also provided is a method for inhibiting polymerase activity comprising contacting polymerase with the hydantoin derivatives of the present invention in an amount sufficient to inhibit the polymerase activity. The inhibition of the polymerase activity by the compounds of the present invention is useful, inter alia, for the treatment and prevention of HIV, AIDS and retrovirus-associate cancer. In one embodiment, the polymerase activity is the polymerase activity of HIV reverse transcriptase. In a particularly preferred embodiment, the polymerase activity is the RNA dependent DNA polymerase (RDDP) activity of reverse transcriptase.

In another embodiment, the invention relates to a method for inhibiting the replication of HIV in a cell comprising contacting an HIV infected cell with an effective amount of the hydantoin derivatives of the present invention, under conditions permitting the uptake of the hydantoin derivative by the HIV infected cell.

The invention also provides a method for treating or preventing HIV and AIDS comprising administering to a subject an effective amount of a hydantoin derivative of the present invention. The method may further comprise administering other compounds useful in the treatment of HIV and AIDS together with the hydantoin derivatives of the invention to provide a combination therapy for the treatment or prevention of HIV and AIDS.

In a further embodiment of the invention, there is provided a method for treating or preventing retrovirus-associated cancer comprising administering to a subject an effective amount of a hydantoin derivative of the present invention.

The present invention also provides a method for screening for candidate hydantoin derivatives having RNase H, polymerase and/or HIV reverse transcriptase modulatory activity.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

The present invention relates to compounds derived from hydantoin. Preferably, the hydantoin compounds of the present invention are of formula I:

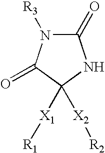

wherein R₁, R₂ and R₃ are independently selected from the group consisting of alkyl, alkenyl, alkynyl, aryl alkyl, alkylaryl, heterocycloalkyl, cycloalkyl, aryl, heteroaryl, any of which may be optionally substituted, and wherein Xᵢ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

The present invention encompasses all tautomeric forms of the compounds of formula I. In the description of this invention, the standard convention of illustrations or naming the compound as the carbonyl structure was used. However, many of the compounds are capable of tautomerizing into the corresponding enol form and these structures are encompassed within this invention.

The compounds of this invention may contain an asymmetric carbon atom and some of the compounds of this invention may contain one or more asymmetric centers and may thus give rise to stereoisomers, such as enantiomers and diastereomers. The stereoisomers of the instant invention are named according to the Cahn-Ingold-Prelog System. While formula I compounds are shown without regard to stereochemistry, the present invention nonetheless includes all the individual possible stereoisomers; as well as the racemic mixtures and other mixtures of R and S stereoisomers (scalemic mixtures which are mixtures of unequal amounts of enantiomers) and pharmaceutically acceptable salts thereof. It should be noted that stereoisomers of the invention having the same relative configuration at a chiral center may nevertheless have different R and S designations depending on the substitution at the indicated chiral center.

The compounds of the current invention may be alkene diastereomers. The alkene diastereomers can be designated using the conventional (E)-(Z) system. This system is well known and understood by those skilled in the art. Where alkene compounds are disclosed without stereospecifity, it is intended that both of the diastereomers are encompassed.

For purposes of this invention the term “alkyl” includes either straight or branched alkyl moieties. The length of a straight alkyl moiety can be from 1 to 12 carbon atoms, but is preferably 1 to 8 carbon atoms. Branched alkyl moieties can contain 3 to 12 carbon atoms, but preferably contain 3 to 8 carbon atoms. These alkyl moieties may be unsubstituted or substituted. The term “alkenyl” refers to a substituted or unsubstitued radical aliphatic hydrocarbon containing one double bond and includes alkenyl moieties of both straight, preferably of 2 to 8 carbon atoms and branched, preferably of 3 to 8 carbon atoms, chains. Such alkenyl moieties may exist in the E or Z configurations; the compounds of this invention include both configurations. The term “alkynyl” includes substituted and unsubstituted alkynyl moieties of both straight chain containing 2 to 8 carbon atoms and branched chain containing 4 to 8 carbon atoms having at least one triple bond. The term “cycloalkyl” refers to substituted or unsubstituted alicyclic hydrocarbon groups having 3 to 12 carbon atoms and includes but is not limited to: cyclopropyl, cyclobutyl, cyclopentyl, cyclohexyl, cycloheptyl, norbornyl, or adamantyl. For purposes of this invention the term “aryl” is defined as an aromatic hydrocarbon moiety and may be substituted or unsubstituted and preferably has 6 to 12 carbon atoms. An aryl moiety may be selected from, but is not limited to, the group consisting of: phenyl, disubstituted chlorophenyl, trisubstituted chlorophenyl, alkoxy substituted phenyl, cyano substituted phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. The substituted aryl, heteroaryl, cycloalkyl, or heterocycloalkyl may be optionally mono-, di-, tri- or tetra-substituted with substituents selected from, but not limited to, the group consisting of alkyl, acyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkylthio, —SO₃H, —SO₂NH₂, —SO₂NHalkyl, —SO₂N(alkyl)₂, —CO₂H, —CO₂— alkyl, CO₂NH₂, CO₂NHalkyl, and —CO₂N(alkyl)₂.

For purposes of this invention the term “heteroaryl” is defined as an aromatic heterocyclic ring system (monocyclic or bicyclic) and may be substituted or unsubstituted where the heteroaryl moieties are five or six membered rings containing 1 to 4 heteroatoms selected from the group consisting of S, N, and O, and include but are not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having one nitrogen atom; (ii) fused to a 5 or 6-membered aromatic (unsaturated) heterocyclic ring having two nitrogen atoms; (iii) fused to a 5-membered aromatic (unsaturated) heterocyclic ring having one nitrogen atom together with either one oxygen or one sulfur atom; or (iv) fused to a 5-membered aromatic (unsaturated) heterocyclic ring having one heteroatom selected from O, N or S. Preferably a heterocycle moiety contains 2 to 9 carbon atoms.

The phrase “linker group” refers a moiety of up to six atoms that connects an R group to the central ring of a compound of formula (I). Preferably the linker group atoms are C, N, or O, any of which may be optionally substituted, and, more preferably, the linker group is either —CH₂CH₂— or —CH₂—.

For the purposes of this invention the term “oxidized” refers to the substitution of the linker group atoms with oxygen, for example a “—CH₂—” moiety can be oxidized to “—CHOH—” or “C(O)—”.

For the purposes of this invention the term “alkoxy” is defined as C₁-C₁₂alkyl-O—; the term “aryloxy” is defined as aryl-O—; the term “heteroaryloxy” is defined as heteroaryl-O—; wherein alkyl, aryl, heteroaryl, cycloalkyl, and heterocycloalkyl are as defined above.

For purposes of this invention the term “arylalkyl” is defined as aryl-C₁-C₆-alkyl, preferably the arylalkyl moiety is comprised of 7-12 carbon atoms. Arylalkyl moieties include benzyl, 1-phenylethyl, 2-phenylethyl, 3-phenylpropyl, 2-phenylpropyl and the like.

For purposes of this invention the term “alkylaryl” is defined as C₁-C₈-alkyl-aryl. Preferably the alkylaryl moiety is comprised of 7-12 carbon atoms.

For purposes of this invention the term “alkylthio” is defined as C₁-C₈-alkyl-S—.

For purposes of this invention “alkoxyalkoxy,” denote an alkoxy group as defined above that is further substituted with an alkoxy group as defined above.

For purposes of this invention “arylthio” and “heteroarylthio,” denote a thio group that is further substituted with an aryl or heteroaryl group as defined above.

For purposes of this invention “phenylalkynyl” is an alkynyl group further substituted with a phenyl group.

The terms “monoalkylamino” and “dialkylamino” refer to moieties with one or two alkyl groups wherein the alkyl chain is 1 to 8 carbons and the groups may be the same or different. The terms monoalkylaminoalkyl and dialkylaminoalkyl refer to monoalkylamino and dialkylamino moieties with one or two alkyl groups (the same or different) bonded to the nitrogen atom which is attached to an alkyl group of 1 to 8 carbon atoms.

“Acyl” is a radical of the formula (C═O)-alkyl or (C═O)-perfluoroalkyl wherein the alkyl radical or perfluoroalkyl radical is 1 to 8 carbon atoms; preferred examples include but are not limited to, acetyl, propionyl, butyryl, trifluoroacetyl.

The term “carbonyl” or “oxo” refers to the radical C(O)—.

Saturated or partially saturated heteroaryl groups are defined in this invention as heterocyclic rings selected from but not limited to the moieties: azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothienyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, tetrahydrothienyl, tetrahydroquinolinyl, and tetrahydroisoquinolinyl.

The term “substituent” is used herein to refer to an atom radical, a functional group radical or a moiety radical that replaces a hydrogen radical on a molecule. Unless expressly stated otherwise, it should assumed that any of the substituents may be optionally substituted with one or more groups selected from: alkyl, halogen, haloalkyl, hydroxyalkyl, nitro, amino, hydroxy, cyano, alkylamino, dialkylamino, alkoxy, haloalkoxy, alkoxyalkyl, alkoxyalkoxy, oxo, alkylthio, mercapto, haloalkylthio, aryl, aryloxy, arylthio, heteroaryl, heteroaryloxy, heteroarylthio, acyl, —CO₂-alkyl, —SO₃H, SO₂NH₂, SO₂NH-alkyl, SO₂NH-(alkyl)₂, CO₂H, CO₂NH₂, CO₂NH-alkyl and CO₂N-(alkyl)₂.

For the purposes of this invention the term “substituted” refers to where a hydrogen radical on a molecule has been replaced by another atom radical, a functional group radical or a moiety radical; these radicals being generally referred to as “substituents.”

In one preferred embodiment the compounds are of formula I where R₁ and R₂ are selected from the group consisting of phenyl, substituted phenyl, alkyl or arylalkyl, and alkylaryl. In another preferred embodiment the compounds are of formula I where R₃ is selected from the group consisting of phenyl and substituted phenyl.

In a further preferred embodiment, the hydantoin compounds of the present invention are of formula I and are selected from the group consisting of the following compounds:
4-ethyl-4-(4-methylphenyl)-4H-imidazole-2,5-diol;
6-[{[{(2,5-dioso-4,4-diphenylimidazolidin-1-yl)acetyl]amino}(phenyl)acetyl]amino}-1-3,3-dimethyl-7-oxo-4-thia-1-azabicyclo[3.2.0]heptane-2-carboxylic acid;
5-methyl-5-phenyl-3-[(2E)-3-phenylprop-2-enoyl]imidazolidine-2,4-dione;
5,5-dimethyl-3-vinyl-imidazolidine-2,4-dione;
3-[2-(3-chloro-4-methylphenyl)-1-methyl-2-oxoethyl]-5-methyl-5-phenyl-2,4-imidazolidinedione;
3-[(2E)-3-(3,4-dichlorophenyl)prop-2-enoyl]-5-methyl-5-phenylimidazolidine-2,4-dione;
3-(3-bromopropyl)-5,5-diphenyl-2,4-imidazolidinedione;
(5R)-5-methyl-5-phenyl-3-{(2E)-3-[4(trifluoromethyl)phenyl]prop-2-enoyl}imidizolidine-2,4-dione;
di(tert-butyl) 8-benzyl-2,4-dioxo-1,3,8-triazaspiro[4.5]decane-1,3-dicarboxylate;
(5S)-5-methyl-5-phenyl-3-{(2E)-3-[4-(trifluoromethyl)phenyl]prop-2-enoyl}imidazolidine-2,4-dione;
3-{(2E)-3-[4-fluoro-3-(trifluoromethyl)phenyl]prop-2-enoyl}-5-methyl-5-phenylimidazolidine-2,4-dione; and
3-(4-chloro-3-methylbenzoyl)-5-methyl-5-phenylimidazolidine-2,4-dione.

In further preferred embodiment, the hydantoin derivatives of the present invention are selected from the group consisting of the following compounds:
(5S)-5-ethyl-5-(4-methylphenyl)imidazolidine-2,4-dione;
5-(3,5-dibromo-2-hydroxy-benzylidene)-imidazolidine-2,4-dione;
5-cyclopropyl-5-(3,4-dimethyl-phenyl)-2,4-imidazolidinedione;
5-(3,5-dichloro-2-hydroxy-benzylidene)-imidazolidine-2,4-dione;
2-benzoyl-10b-phenyl-6,10b-dihydro-5H-imidazo[5,1-a]isoquinoline-1,3-dione; and
5-(4-bromo-phenyl)-5-ethyl-imidazolidine-2,4-dione.

The invention also provides for methods of synthesis of the compounds of the present invention. For example, the hydantoins of the present invention may be prepared according to the scheme set forth below:

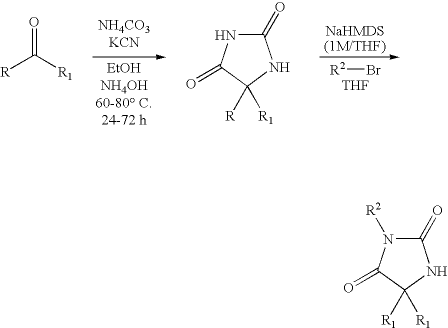

In a preferred embodiment, the process for preparing a hydantoin derivative of the present invention comprises (a) producing the compound of formula X:

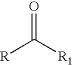

(b) producing from the compound of formula X, via the condensation of a ketone with potassium cyanide and ammonium carbonate, the compound of formula XI:

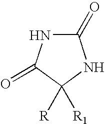

(c) producing from the compound of formula XI, via N-alkylation, the compounds of formula I:

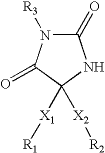

wherein R₁, R₂ and R₃ are independently selected from the group consisting of alkyl, alkenyl, alkynyl, aryl alkyl, alkylaryl, heterocycloalkyl, cycloalkyl, aryl, heteroaryl, any of which may be optionally substituted,

and wherein Xᵢ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

The compounds of the present invention modulate, and preferably inhibit, RNase H nuclease activity. RNase H is an enzyme responsible for the removal of RNA primers from leading and lagging strands during DNA synthesis. It is an important enzyme for the replication of bacterial, viral and human genomes. HIV reverse transcriptase has an RNase H domain at the C-terminus of its p66 subunit. Accordingly, the compounds of the present invention modulate, and preferably inhibit, HIV reverse transcriptase. The ability of the compounds of the present invention to inhibit RNase H, and more particularly HIV reverse transcriptase, may be measured or determined by any means known in the art.

Preferably, the RNase H/HIV reverse transcriptase modulatory activity of the compounds of the present invention may be determined by the methods described in a copending U.S. provisional patent Application No. 60/436,125, filed Dec. 19, 2002 and PCT Publication No. WO 2004/059012, filed Dec. 22, 2003, published Jul. 15, 2004, entitled ASSAY FOR RNase ACTIVITY of Olson et al., incorporated herein by reference in their entirety. Specifically, the modulatory activity of a hydantoin derivative of the present invention may be determined by hybridizing a target nucleic acid to a fluorescently labeled oligonucleotide probe complementary to the target nucleic acid and containing a fluorophor at one terminus and a quenching group at the other terminus to obtain a probe-target hybrid, wherein (i) the unhybridized probe adopts a conformation that places the fluorophor and quencher in such proximity that the quencher quenches the fluorescent signal of the fluorophor, and (ii) the formation of the probe-target hybrid causes sufficient separation of the fluorophor and quencher to reduce quenching of the fluorescent signal of the fluorophor. Next, a first and second sample containing the probe-target hybrid are prepared. The probe-target hybrid of the first sample is then contacted with an RNase H enzyme (such as HIV reverse transcriptase) in an amount sufficient to selectively cleave the target nucleic acid and thereby release the intact probe. The probe-target hybrid of the second sample is also contacted with the RNase H enzyme in an amount sufficient to selectively cleave the target nucleic acid and thereby release the intact probe in the presence of a hydantoin derivative of the present invention. The release of the probe in each sample may then be detected by measuring the decrease in the fluorescent signal of the fluorophor as compared to the signal of the probe-target hybrid. A comparison of the rate of the decrease in the fluorescent signal of the fluorophor in the two samples is made to determine whether there is a difference in the rate of the decrease in the two samples. A difference in the rate of decrease in the samples indicates that the hydantoin compound is a modulator of RNase H/HIV reverse transcriptase. This method is also useful to identify hydantoin derivatives of the present invention, wherein candidate hydantoin derivatives are screened for their ability to modulate RNase/HIV reverse transcriptase activity.

The method of the present invention for modulating, and preferably inhibiting, the nuclease activity of RNase, comprises contacting RNase, either in vitro or in vivo, with the compounds of the present invention. The RNase H modulatory activity, and particularly inhibitory activity, of the compounds of the present invention indicates that they are useful for inhibiting the replication of HIV in a cell infected with HIV. It further indicates that the compounds are useful in the prevention and treatment of HIV and AIDS.

In addition, the compounds of the present invention may be useful for treating other microbial infections, including bacterial and viral infections, wherein the bacteria or virus relies on RNase H nuclease activity for replication.

The compounds may further be useful for treating certain cancers, and particularly retrovirus associated adenocarcinomas, such as breast cancer. See U.S. Pat. No. 5,223,490, incorporated herein by reference in its entirety.

The hydantoin compounds of the present invention preferably inhibit RNase H and HIV reverse transcriptase with IC50 values of 1 to 100 μM. In one embodiment, the compounds of the present invention inhibit HIV reverse transcriptase with the IC50 values shown in Table I below:

TABLE I

| Compound | MW | IC50 |
|---|---|---|
| (5S)-5-ethyl-5-(4-methylphenyl)imidazolidine-2,4-dione | 218.3 | 2 |
| 4-ethyl-4-(4-methylphenyl)-4H-imidazole-2,5-diol | 218.3 | 4 |
| 6-[{[{(2,5-dioso-4,4-diphenylimidazolidin-1- | 641.7 | 4 |
| yl)acetyl]amino}(phenyl)acetyl]amino}-3,3-dimethyl-7- | | |
| oxo-4-thia-1-azabicyclo[3.2.0]heptane-2-carboxylic acid | | |
| 5-(3,5-dibromo-2-hydroxy-benzylidene)-imidazolidine- | 362.0 | 13 |
| 2,4-dione | | |
| 5-cyclopropyl-5-(3,4-dimethyl-phenyl)-2,4- | 244.3 | 41 |
| imidazolidinedione | | |
| 5-methyl-5-phenyl-3-[(2E)-3-phenylprop-2- | 320.3 | 46 |
| enoyl]imidazolidine-2,4-dione | | |
| 5,5-dimethyl-3-vinyl-imidazolidine-2,4-dione | 154.2 | 50 |
| 3-[2-(3-chloro-4-methylphenyl)-1-methyl-2-oxoethyl]-5- | 370.8 | 51 |
| methyl-5-phenyl-2,4-imidazolidinedione | | |
| 3-[(2E)-3-(3,4-dichlorophenyl)prop-2-enoyl]-5-methyl-5- | 389.2 | 52 |
| phenylimidazolidine-2,4-dione | | |
| 3-(3-bromopropyl)-5,5-diphenyl-2,4-imidazolidinedione | 373.2 | 56 |
| 5-(3,5-Dichloro-2-hydroxy-benzylidene)-imidazolidine- | 273.1 | 57 |
| 2,4-dione | | |
| (5R)-5-methyl-5-phenyl-3-{(2E)-3- | 388.3 | 58 |
| [4(trifluoromethyl)phenyl]prop-2-enoyl]imidizolidine- | | |
| 2,4-dione | | |
| 2-benzoyl-10b-phenyl-6,10b-dihydro-5H-imidazo[5,1- | 382.4 | 63 |
| a]isoquinoline-1,3-dione | | |
| 5-(4-bromo-phenyl)-5-ethyl-imidazolidine-2,4-dione | 283.1 | 64 |
| di(tert-butyl) 8-benzyl-2,4-dioxo-1,3,8- | 549.5 | 87 |
| triazaspiro[4.5}decane-1,3-dicarboxylate | | |
| (5S)-5-methyl-5-phenyl-3-{(2E)-3-[4- | 388.3 | 98 |
| (trifluoromethyl)phenyl]prop-2-enoyl}imidazolidine-2,4- | | |
| dione | | |
| 3-{(2E)-3-[4-fluoro-3-(trifluoromethyl)phenyl]prop-2- | 406.3 | 99 |
| enoyl}-5-methyl-5-phenylimidazolidine-2,4-dione | | |
| 3-(4-chloro-3-methylbenzoyl)-5-methyl-5- | 342.8 | 100 |
| phenylimidazolidine-2,4-dione | | |

The method of the present invention for modulating, and preferably inhibiting, polymerase activity, comprises contacting polymerase, either in vitro or in vivo, with the compounds of the present invention. The polymerase modulatory activity, and particularly inhibitory activity, of the compounds of the present invention indicates that they are useful for inhibiting the replication of HIV in a cell infected with HIV. It further indicates that the compounds are useful in the prevention and treatment of HIV and AIDS.

In addition, the compounds of the present invention may be useful for treating other viral infections, wherein the virus relies on RDDP polymerase activity for replication.

The compounds may further be useful for treating certain cancers, and particularly retrovirus associated adenocarcinomas, such as breast cancer. See U.S. Pat. No. 5,223,490, incorporated herein by reference in its entirety.

The hydantoin compounds of the present invention preferably inhibit polymerase and HIV reverse transcriptase with IC50 values of 1 to 300 μM. In one embodiment, as described further in Example 3 below, the compounds of the present invention inhibit the polymerase activity of HIV reverse transcriptase with the IC50 values shown in Table II below:

TABLE II

| Compound | MW | IC50 |
|---|---|---|
| (5S)-5-ethyl-5-(4-methylphenyl)imidazolidine-2,4-dione | 218.3 | >180 |
| 4-ethyl-4-(4-methylphenyl)-4H-imidazole-2,5-diol | 218.3 | >184 |
| 6-[{[{(2,5-dioso-4,4-diphenylimidazolidin-1- | 641.7 | >62 |
| yl)acetyl]amino}(phenyl)acetyl]amino}-3,3-dimethyl-7- | | |
| oxo-4-thia-1-azabicyclo[3.2.0]heptane-2-carboxylic acid | | |
| 5-(3,5-dibromo-2-hydroxy-benzylidene)-imidazolidine- | 362.0 | >111 |
| 2,4-dione | | |
| 2,4-Imidazolidinedione,5-cyclopropyl-5-(3,4-dimethyl- | 244.3 | >122 |
| phenyl)-CA | | |
| -methyl-5-phenyl-3-[(2E)-3-phenylprop-2- | 320.3 | >260 |
| enoyl]imidazolidine-2,4-dione | | |
| 5,5-dimetyl-3-vinyl-imidazolidine-2,4-dione | 154.2 | >107 |
| 3-[2-(3-chloro-4-metylphenyl)-1-methyl-2-oxoethyl]-5- | 370.8 | >103 |
| methyl-5-phenyl-2,4-imidazolidinedione | | |
| 3-[(2E)-3-(3,4-dichlorophenyl)prop-2enoyl]-5-methyl-5- | 389.2 | >107 |
| phenylimidazolidine-2,4-dione | | |
| 2,4-Imidazolidinedione,3-(3-bromopropyl)-5,5- | 373.2 | >146.5 |
| diphenyl-CA | | |
| 5-(3,5-Dichloro-2-hydroxy-benzylidene)-imidazolidine- | 273.1 | >102 |
| 2,4-dione | | |
| (5R)-5-methyl-5-phenyl-3-{(2E)-3- | 388.3 | >105 |
| [4(trifluoromethyl)phenyl]prop-2-enoyl}imidisolidine- | | |
| 2,4-dione | | |
| 2-benzoyl-10b-phenyl-6,10b-dihydro-5H-imidazo[5,1- | 382.4 | nd |
| a]isoquinoline-1,3-dione | | |
| 5-(4-bromo-phenyl)-5-ethyl-imidazolidine-2,4-dione | 283.1 | nd |
| di(tert-butyl) 8-benzyl-2,4-dioxo-1,3,8- | 549.5 | >103 |
| triazaspiro[4.5}decane-1,3-dicarboxylate | | |
| (5S)-5-methyl-5-phenyl-3-{(2E)-3-[4- | 388.3 | >100 |
| (trifluoromethyl)phenyl]prop-2-enoyl}imidazolidine-2,4- | | |
| dione | | |
| 3-{(2E)-3-[4-fluoro-3-(trifluoromethyl)phenyl]prop-2- | 406.3 | >115 |
| enoyl}-5-methyl-5phenylimidazolidine-2,4-dione | | |
| 3-(4-chloro-3-methylbenzoyl)-5-methyl-5- | 342.8 | 100 |
| phenylimidazolidine-2,4-dione | | |

If an inventive compound is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

A “prodrug” is intended to mean a compound that is converted under physiological conditions or by solvolysis or metabolically to a specified compound that is pharmaceutically active. A prodrug may be a derivative of one of the compounds of this invention that contains a moiety, such as for example —CO₂R, —PO(OR)₂ or —C═NR, that may be cleaved under physiological conditions or by solvolysis. Any suitable R substituent may be used that provides a pharmaceutically acceptable solvolysis or cleavage product. A prodrug containing such a moiety may be prepared according to conventional procedures by treatment of a compound of this invention containing, for example, an amido, carboxylic acid, or hydroxyl moiety with a suitable reagent. A “pharmaceutically active metabolite” is intended to mean a pharmacologically active compound produced through metabolism in the body of a specified compound. Prodrugs and active metabolites of compounds of this invention of the above-described Formulas may be determined using techniques known in the art, for example, through metabolic studies. See, e.g., “Design of Prodrugs,” (Bundgaard, ed.), 1985, Elsevier Publishers B.V., Amsterdam, The Netherlands. A “pharmaceutically acceptable salt” is intended to mean a salt that retains the biological effectiveness of the free acids and bases of a specified compound and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates (mesylates), propanesulfonates, naphthalene-1-sulfonates, naphthalene-2-sulfonates, mandelates and the like. In the case of compounds or salts that are solids it is understood by those skilled in the art that the inventive compounds or salts may exist in different crystal forms, all of which are intended to be within the scope of the present invention and specified formulas.

The present invention provides for pharmaceutical compositions comprising the compounds of the present invention. Pharmaceutical compositions of the present invention comprise an effective amount of a hydantoin derivative of the present invention or pharmaceutically acceptable salt thereof, dissolved and/or dispersed in a pharmaceutically acceptable carrier and/or aqueous medium.

The phrases “physiologically, pharmaceutically and/or pharmacologically acceptable” refer to molecular entities and/or compositions that do not produce an adverse, allergic and/or other untoward reaction when administered to an animal.

As used herein, “physiologically and/or pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards.

The active compounds may generally be formulated for parenteral administration, e.g., formulated for injection via the intravenous, intramuscular, sub-cutaneous, intralesional, and/or even intraperitoneal routes. The preparation of pharmaceutical compositions that contain a therapeutically effective amount of the hydantoin derivatives of the invention or pharmaceutically acceptable salts thereof as an active component and/or ingredient will be known to those of skill in the art in light of the present disclosure.

Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils.

Hydantoin derivatives of the present invention can be formulated into a composition in a neutral and/or salt form. Pharmaceutically acceptable salts, include the acid addition salts and/or which are formed with inorganic acids such as, for example, hydrochloric and/or phosphoric acids, and/or such organic acids as acetic, oxalic, tartaric, mandelic, and/or the like.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

The hydantoin compounds of the present invention may be formulated within a therapeutic mixture to comprise about 0.01 to about 100 milligrams/kilogram per dose. Multiple doses can also be administered, as well as combinations of the hydantoins with other agents useful for the treatment of HIV, AIDS, and retrovirus-associated cancers, such as adenocarcinomas of the breast. Some variation in dosage will necessarily occur depending on the condition of the subject being treated. The person responsible for administration will, in any event, determine the appropriate dose for the individual subject.

Various routes of administration are contemplated for various disease types. For HIV and AIDS, given that most cells of the body are infected, systemic delivery is contemplated. Similarly, for retrovirus-associated cancer, for practically any tumor, systemic delivery is contemplated. This will prove especially important for attacking microscopic or metastatic cancer. Where discrete tumor mass may be identified, a variety of direct, local and regional approaches may be taken.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes. Cremes may be useful for prevention the transmission of HIV and may be used in conjunction with condoms to further ensure that HIV is not transmitted.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra.

In certain embodiments of the present invention, the use of lipid formulations and/or nanocapsules is contemplated for the introduction of the hydantoin derivatives of the present invention or pharmaceutically acceptable salts thereof into host cells. Lipid formulations and nanocapsules may be prepared by methods well known in the art.

As noted above, in order to increase the effectiveness of the hydantoin derivatives of the present invention, it may be desirable to combine these compositions with other agents effective in the treatment of the target disease. Other treatments for HIV and AIDS may include, but are not limited to, AZT (zidovudine, Retrovir), ddI (didanosine, Videx), 3TC (lamivudine, Epivir), d4T (stavudine, Zerit), abacavir (Ziagen), ddC (zalcitabine, Hivid), AZT and 3TC in a single combined pill called Combivir, AZT, 3TC and abacavir in a single combined pill called Trizivir, Sustiva, nevirapine (Viramune), delavirdine (Rescriptor), and Tenofovir (Viread).

For retrovirus-associated cancer, additional anti-cancer agents may be administered. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the hydantoin derivatives of the present invention and other agent(s) at the same time. This may be achieved by contacting the cell with a single composition or pharmacological formulation that includes both agents, or by contacting the cell with two distinct compositions or formulations, at the same or different time, wherein one composition includes the hydantoin and the other includes the second agent(s).

### EXAMPLES

The following examples are offered to illustrate, but not to limit the present invention.

### Example 1

### Preparation of (5S)-5-ethyl-5-(4-methyl phenyl)imidazolidine-2,4-dione

A mixture of 4′-methylpropiophenone (5.0 g), ammonium bicarbonate (9.6 g) and potassium cyanide (2.6 g) in ethanol (13 mL)/ammonium hydroxide (10 mL) was warmed up to 70° C. and stirred overnight in a sealed vessel. After the reaction mixture was cooled to ambient conditions, water (10 mL) was added and the reaction mixture stirred for 1 hour. The reaction mixture was filtered and washed with water (thrice), ether (twice) and dried under vacuum to give 6.25 g (85%) of the title compound as a white solid. (M+H)⁺-219.

### Example 2

### Preparation of 4-ethyl-4-(4-methylphenyl)-4H-imidazole-2,5-diol; M+H⁺-219

3-[2-(3-chloro-4-methylphenyl)-1-methyl-2-oxoethyl]-5-methyl-5-phenyl-2,4-imidazolidinedione; M+H⁺-371;
3-[(2E)-3-(3,4-dichlorophenyl)prop-2-enoyl]-5-methyl-5-phenylimidazolidine-2,4-dione; M−H⁻-387;
(5R)-5-methyl-5-phenyl-3-{(2E)-3-[4(trifluoromethyl)phenyl]prop-2-enoyl}imidizolidine-2,4-dione; M−H⁺-387;
6-[{[{(2,5-dioso-4,4-diphenylimidazolidin-1-yl)acetyl]amino}(phenyl)acetyl]amino}-3,3-dimethyl-7-oxo-4-thia-1-azabicyclo[3.2.0]heptane-2-carboxylic acid;
5-methyl-5-phenyl-3-[(2E)-3-phenylprop-2-enoyl]imidazolidine-2,4-dione;
5,5-dimethyl-3-vinyl-imidazolidine-2,4-dione;
3-(3-bromopropyl)-5,5-diphenyl-2,4-imidazolidinedione;
di(tert-butyl) 8-benzyl-2,4-dioxo-1,3,8-triazaspiro[4.5]decane-1,3-dicarboxylate;
(5S)-5-methyl-5-phenyl-3-{(2E)-3-[4-(trifluoromethyl)phenyl]prop-2-enoyl}imidazolidine-2,4-dione;
3-(4-chloro-3-methylbenzoyl)-5-methyl-5-phenylimidazolidine-2,4-dione;
5-(3,5-dibromo-2-hydroxy-benzylidene)-imidazolidine-2,4-dione;
5-cyclopropyl-5-(3,4-dimethyl-phenyl)-2,4-imidazolidinedione;
5-cyclopropyl-5-(3,4-dimethyl-phenyl)-2,4-imidazolidinedione;
5-(3,5-dichloro-2-hydroxy-benzylidene)-imidazolidine-2,4-dione;
2-benzoyl-10b-phenyl-6,10b-dihydro-5H-imidazo[5,1-a]isoquinoline-1,3-dione; and
5-(4-bromo-phenyl)-5-ethyl-imidazolidine-2,4-dione.

### Example 3

### Procedures for Determining the Polymerase Inhibitory Activity of the Hydantoin Compounds of the Invention

### Materials and Methods:

The RNA dependent DNA polymerase (RDDP) activity of HIV RT was evaluated using polyrA-oligodT₁₂₋₁₈ as the template-primer allowing for TTP incorporation (Telesnitsky, A., Blain, S, and Goff, S. P. (1995) Methods in Enzymology 262, 347-362 and Goff, S, Traktman, P., and Baltimore, D (1981) J. Virology 38, 239). The Michaelis Constants for HIV RT RDDP were first determined for the two substrates TTP and polyrA-oligodT₁₂₋₁₈ independently. The Kₘ-values for TTP and polyrA-oligodT₁₂₋₁₈ were determined to be 7.1 μM and 5.4 nM, respectively.

### Materials Used:

HIV Reverse transcriptase (RT) 66/p51 at a concentration of 10800 units/mg (19.6 μM following stabilization in 50% glycerol) was obtained from Worthington. The template primer used was polyrA-oligodT12-18 at 4.47 μM as substrate was obtained from Pharmacia, as well as TTP (thymidine tri-phosphate), which was stored at a concentration of 1 mM. ³³P TTP at 10 μCi/μl (3000 Ci/mmol) and 3.3 μM was obtained from NEN/DuPont. A 5×HIV RT buffer was prepared with the 1× final concentration being 50 mM Tris-HCl (pH 8.5), 6 mM MgCl₂, 80 mM KCl, 1 mM DTT (dithiotrheitol), 0.05% Triton X-100, 0.05 mg/ml BSA (bovine serum albumin). The wash buffer consisted of 0.5 M Na₂HPO₄ (pH 7.0). Filter plates were obtained from Millipore Corp. The scintillant used was Optiphase Supermix™ from Wallac/Perkin Elmer.

### Methods:

A 25 μl reaction was generated from the reagents above in the following manner: An enzyme mix (consisting of 2.5× reaction buffer, 100% DMSO, and 25 fmol of HIV RT) and a substrate mix (consisting of 0.1625 mM TTP, 0.00725 μM ³³P TTP [0.00725 μCi] and 0.015 μM polyrA-dT was generated. Both mixes were stable for up to 1 hour at room temperature. The enzyme, reverse transcriptase was added to the enzyme mix after the other constituents of the enzyme mix were made homogeneous. For the reaction, 5 μl of test compound (or 15% DMSO) was mixed with 10 μl enzyme mix and 10 μl substrate mix and the final mixture was incubated for 2 hours at room temperature. EDTA controls contained 10 fmol enzyme and was used to determine the non-specific retention of the radio-labeled nucleotide in the filter plate, i.e. it is a mock reaction. The reaction was stopped after 2 hours by the addition of 100 μl of 50 mM EDTA. The filter plates were prewashed with 200 μl of wash buffer using a vacuum applied to the filter. 100 μl of each sample was filtered through the filter plates and then they were washed 3 times with 200 μl of wash buffer. 1 microliter of reaction mix was spotted onto a filter to determine specific activity of the reaction mix. The plates were allowed to dry for 30 minutes to 60 minutes. Scintillant was added and the counts per minute were determined in a Wallac Micro-Beta™ counter.

One unit of HIV RT is defined as that amount of enzyme that results in the incorporation of 1 nmol of TMP (thymidine mono-phosphate) into an acid insoluble precipitate in 10 minutes at 37° C. using polyrA oligodT₁₂₋₁₈ as the template primer (Worthington Enzyme Corporation Catalogue year 2001).

An enzyme mix and a substrate mix was generated. To prepare the enzyme mix, the enzyme was added to the enzyme mix last to ensure it was buffered and maintained in a reduced state (presence of dithiothreitol, DTT). It is critical not to vortex the enzyme mix after the addition of enzyme. Rather, the enzyme was mixed into solution by gentle inversion or pipetting or mixing, not by vortexing. To generate a homogeneous mixture of the substrate solution gentle vortexing was than used. The enzyme solution was added to plates containing compounds. 5 ul of 15% DMSO was added to the non-compound containing samples. DMSO at a concentration of 3% stimulates HIV RT RDDP activity up to 3-fold. Without the addition of DMSO in the positive control samples an under-estimate of the inhibitory activity of the compound being assayed is obtained. The enzyme was then incubated with the compound for 15 min at room temperature (˜23° C.) prior to the addition of the substrate mix. The enzyme was allowed to incubate with the substrate for 2 hour at room temperature (˜23° C.). Under these conditions enzyme reaction was linear for >4 hours and utilizes less than 7% of the available substrates (TTP and polyrA-oligodT₁₂₋₁₈). The assays were stopped by the addition of 100 ul of 50 mM EDTA and 100 ul of each sample was subjected to filtration in the Millipore DE MADEN OB50 plates. These plates were washed to remove unincorporated radiolabeleled nucleotides, dried and subjected to counts per minute (cpm) measurement in the Wallac Micro-Beta™p counter after the addition of scintillant. The quantity of TTP incorporated was then determined by the specific activity (S.A.) of the reaction mix, as discussed in the results section below, to ensure that less than 10% of the available substrates were consumed in the reaction and ensured linearity of the enzyme reaction. In addition, a mock reaction was included as a control. This reaction contained all of the assay components but contained the addition of 100 ul of 50 mM EDTA at the initiation of the reaction. This mock reaction control determined the quantity of background counts (cpm) in the reaction.

### Analysis of Results:

IC₅₀-values<10 ug/ml or 10 μM were considered active (See Table II above for IC₅₀-values.

Calculation

The instrument used for quantitation was a Wallac Micro-Beta linked to a Windows based compatible desktop computer. The specific activity (S.A.) of the reaction mix was defined as cpm/pmol of TTP in the mix. (cpm counts per minute in scintillation counting.) As noted above, 1 ul from a reaction was spotted in triplicate onto a filter the Millipore DE MADEN OB50 plates. This filter was not subjected to the washing procedure. It was used to accurately reflect the concentration of radioactivity per pmol of nucleotide in the reaction mixture. To determine specific activity, the following calculation was used S.A.=total cpm per ul divided by pmol TTP per ul, which provides cpm/pmol of TTP in the reaction. The Kₘ-value was ˜7 μM, 6.5 μM concentration of TTP is used in the reaction.

In the reaction the incorporation of nucleotide by cpm was measured. This was converted to pmol of nucleotide incorporated by dividing the total number of cpms in the reaction by the S.A. of that reaction

Sample Calculation

The pmol of nucleotides incorporated in reaction X was calculated as follows: Cpm of reaction X divided by a given unit of time which equals pmol of TTP incorporated in reaction X in a given unit time. The S.A. of the reaction for the HIV RT assay was: S.A.=10,000 cpm/(6.5 pmol/ul)=1538.46 cpm/pmol. The final concentration of TTP was 6.5 uM (6.5 pmol/ul). 5500 cpms were measured in reaction X, but only 100 ul of 125 ul of the reaction was transferred to the filter plate from the reaction plate. The background retention of radiolabeled nucleotide was determined to be 125 cpm (5500 cpm-125 cpm is 5375 cpm; =5375 cpm×(125/100)=6718.75 cpm is the total for reaction X=6718.75 cpm/(1538.46 cpm/pmol)=4.36 pmol of TTP was incorporated. The IC₅₀-values for the compounds of the invention are shown in Table II above.

As reference compounds, Efavirenz was used which had an IC₅₀-value of <1 μM and AZT was used, which had an IC₅₀-value of <0.2 μM.

The above-described invention may be varied in ways obvious to those skilled in this art. All such modifications are intended to be included within the scope of this invention. This invention is not to be limited except as set forth in the claims which follow.

1. A compound of formula I: 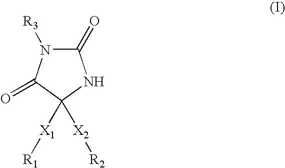
wherein R₁, R₂ and R₃ are independently selected from the group consisting of alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

2. The compound of claim 1, wherein
R₁, R₂ and R₃ are independently selected from the group consisting of C₁-C₈ alkyl, C₃-C₁₂ cycloalkyl, C₅-C₁₂ aryl, C₂-C₉ heteroaryl, any of which may be optionally substituted.

3. The compound of claim 1 wherein R₁ and R₂ are independently selected from the group consisting of an alkyl, an aryl, an alkylaryl, and an arylakyl.

4. The compound of claim 1, wherein R₁ and R₂ are cycloalkyl.

5. The compound of claim 1, wherein R₃ is an aryl.

6. The compound of claim 1, wherein R₁ is an aryl selected from the group consisting of phenyl and substituted phenyl.

7. The compound of claim 1, wherein R₂ is an aryl selected from the group consisting of phenyl and substituted phenyl.

8. The compound of claim 1, wherein R₃ is an aryl selected from the group consisting of phenyl and substituted phenyl.

9. The compound of claim 1, wherein the compound is selected from the group consisting of:
6-{[{[(2,5-dioxo-4,4-diphenylimidazolidin-1-yl)acetyl]amino}(phenyl)acetyl]amino}-3,3-dimethyl-7-oxo-4-thia-1-azabicyclo[3.2.0]heptane-2-carboxylic acid;
5-methyl-5-phenyl-3-[(2E)-3-phenylprop-2-enoyl]imidazolidine-2,4-dione;
5,5-dimethyl-3-vinyl-imidazolidine-2,4-dione;
3-[2-(3-chloro-4-methylphenyl)-1-methyl-2-oxoethyl]-5-methyl-5-phenyl-2,4-imidazolidinedione;
3-[(2E)-3-(3,4-dichlorophenyl)prop-2-enoyl]-5-methyl-5-phenylimidazolidine-2,4-dione;
3-(3-bromopropyl)-5,5-diphenyl-2,4-imidazolidinedione;
(5R)-5-methyl-5-phenyl-3-{(2E)-3-[4(trifluoromethyl)phenyl]prop-2-enoyl}imidazolidine-2,4-dione;
(5S)-5-methyl-5-phenyl-3-{(2E)-3-[4-(trifluoromethyl)phenyl]prop-2-enoyl}imidazolidine-2,4-dione;
3-{(2E)-3-[4-fluoro-3-(trifluoromethyl)phenyl]prop-2-enoyl}-5-methyl-5-phenylimidazolidine-2,4-dione; and
3-(4-chloro-3-methylbenzoyl)-5-methyl-5-phenylimidazolidine-2,4-dione;
or a prodrug, a pharmaceutically acceptable salt, or a pharmaceutically active metabolite thereof.

10. A method for inhibiting RNase nuclease activity comprising contacting RNase with an effective amount of a hydantoin of formula I: 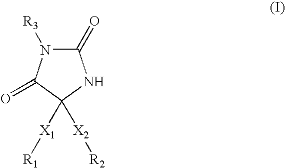
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

11. A method for inhibiting polymerase activity comprising contacting polymerase with an effective amount of a hydantoin of formula I: 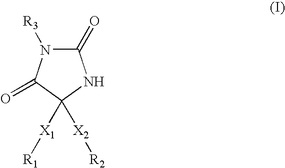
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

12. A method for inhibiting HIV reverse transcriptase activity comprising contacting the HIV reverse transcriptase with an effective amount of a hydantoin compound of formula I: 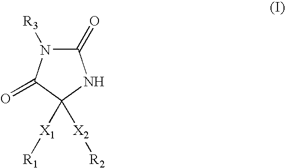
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

13-20. (canceled)

21. A method for inhibiting HIV replication in a cell infected with HIV comprising contacting the cell with an effective amount of a hydantoin compound of formula I: 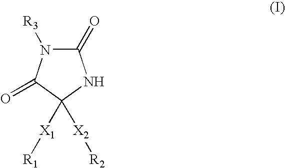
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

22. A method for preventing or treating HIV or AIDS comprising administering to a subject an effective amount of a hydantoin compound of formula I: 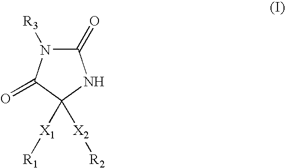
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

23. A method for preventing or treating retrovirus-associated cancer comprising administering to a subject an effective amount of a hydantoin compound of formula I: 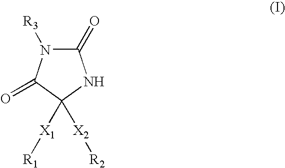
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

24. A method for inhibiting the growth of a retrovirus-associate cancer cell comprising administering to the cell an effective amount of a hydantoin compound of formula I: 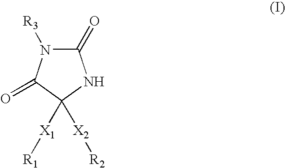
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

25-32. (canceled)

33. The methods of any one of claim 10, 11, 12, 21, 22, 23 or 24, further comprising co-administering an anti-HIV or anti-AIDs compound.

34. A method for screening for a candidate hydantoin compound having RNase nuclease modulatory activity comprising:
(a) hybridizing a target nucleic acid to a fluorescently labeled oligonucleotide probe complementary to the target nucleic acid and containing a fluorophor at one terminus and a quenching group at the other terminus to form a probe-target hybrid, wherein (i) the unhybridized probe adopts a conformation that places the fluorescent signal of the fluorophor and quencher in such proximity that the quencher quenches the fluorescent signal of the fluorophor and (ii) the formation of the probe-target hybrid causes sufficient separation of the fluorophor and quencher to reduce quenching of the fluorescent signal of the fluorophor;
(b) preparing a first and second sample containing the probe-target hybrid;
(c) contacting the probe-target hybrid of the first sample with the RNase in an amount sufficient to selectively cleave the target nucleic acid and thereby release the intact probe;
(d) contacting the probe-target hybrid of the second sample with the RNase in an amount sufficient to selectively cleave the target nucleic acid and thereby release the intact probe in the presence of the candidate hydantoin;
(e) detecting the release of the probe in the first and second sample by measuring the decrease in the fluorescent signal of the fluorophor as compared to the signal of the probe-target hybrid; and
(f) comparing the rate of the decrease in the fluorescent signal of the fluorophor in the first and second sample, wherein a difference in the rate is indicative of the ability of the candidate hydantoin to modulate the RNase nuclease activity.

35. A method for screening for a candidate hydantoin compound having HIV reverse transcriptase modulatory activity comprising:
(a) hybridizing a target nucleic acid to a fluorescently labeled oligonucleotide probe complementary to the target nucleic acid and containing a fluorophor at one terminus and a quenching group at the other terminus to form a probe-target hybrid, wherein (i) the unhybridized probe adopts a conformation that places the fluorescent signal of the fluorophor and quencher in such proximity that the quencher quenches the fluorescent signal of the fluorophor and (ii) the formation of the probe-target hybrid causes sufficient separation of the fluorophor and quencher to reduce quenching of the fluorescent signal of the fluorophor;
(b) preparing a first and second sample containing the probe-target hybrid;
(c) contacting the probe-target hybrid of the first sample with the HIV reverse transcriptase in an amount sufficient to selectively cleave the target nucleic acid and thereby release the intact probe;
(d) contacting the probe-target hybrid of the second sample with the HIV reverse transcriptase in an amount sufficient to selectively cleave the target nucleic acid and thereby release the intact probe in the presence of the candidate hydantoin;
(e) detecting the release of the probe in the first and second sample by measuring the decrease in the fluorescent signal of the fluorophor as compared to the signal of the probe-target hybrid; and
(f) comparing the rate of the decrease in the fluorescent signal of the fluorophor in the first and second sample, wherein a difference in the rate is indicative of the ability of the candidate hydantoin to modulate the nuclease activity of the RNase.

36. The method of claim 34 or 35, wherein the candidate hydantoin compound is of formula I: 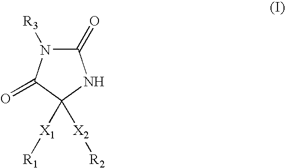
wherein R₁, R₂ and R₃ are independently selected from the group consisting of hydrogen, alkyl, alkenyl, alkynyl, aryl alkyl, alkylaryl, heterocycloalkyl, cycloalkyl, aryl, heteroaryl, any of which may be optionally substituted,
and wherein X₁ and X₂ are a bond or a linker group consisting of 1 to 6 atoms selected from the group consisting of C, N or O, any of which may be optionally substituted, saturated, partially unsaturated or fully unsaturated; or a prodrug, a pharmaceutically acceptable salt or a pharmaceutically active metabolite thereof.

37-43. (canceled)

44. A pharmaceutical composition comprising a pharmaceutically effective amount of a compound of claim 1, 45, 46, or 47 and a pharmaceutically acceptable carrier.

45. 4-ethyl-4-(4-methylphenyl)-4H-imidazole-2,5-diol.

46. Di(tert-butyl) 8-benzyl-2,4-dioxo-1,3,8-triazaspiro[4.5]decane-1,3-dicarboxylate.

47. 2-benzoyl-10b-phenyl-6,10b-dihydro-5H-imidazo[5,1-a]isoquinoline-1,3-dione.
